(19) United States
(12) Patent Application Publication
(10) Pub. No.: US 2005/0003424 A1
(43) Pub. Date: Jan. 6, 2005

(54) Testis-specific receptor

(76) Inventors: James W. Baumgartner, Thousand Oaks, CA (US); Theresa M. Farrah, Cairo (EG); Donald C. Foster, Lake Forest Park, WA (US); Frank J. Grant, Seattle, WA (US); Patrick J. O'Hara, Seattle, WA (US)

Correspondence Address: FITZPATRICK CELLA HARPER & SCINTO, 30 ROCKEFELLER PLAZA, NEW YORK, NY, 10112, US

(21) Appl. No.: 10/887,865

(22) Filed: Jul. 12, 2004

Related U.S. Application Data
(63) Continuation of application No. 09/090,867, filed on Jun. 4, 1998, now abandoned.
Application No. 09/090,867 is a division of application No. 08/815,773, filed on Mar. 12, 1997, now abandoned.
(60) Provisional application No. 60/013,345, filed on Mar. 13, 1996.

(51) Int. Cl. C12Q 1/68; C07H 21/04; C07K 14/705

(52) U.S. Cl. 435/6; 435/69.1; 435/320.1; 435/325; 530/350; 536/23.5

(57) ABSTRACT

Novel receptor polypeptides, polynucleotides encoding the polypeptides, and related compositions and methods are disclosed. The polypeptides comprise an extracellular domain of a cell-surface receptor that is expressed in testis cells. The polypeptides may be used within methods for detecting ligands that promote the proliferation and/or differentiation of testis cells, and may also be used in the development of male-specific contraceptives and infertility treatments.

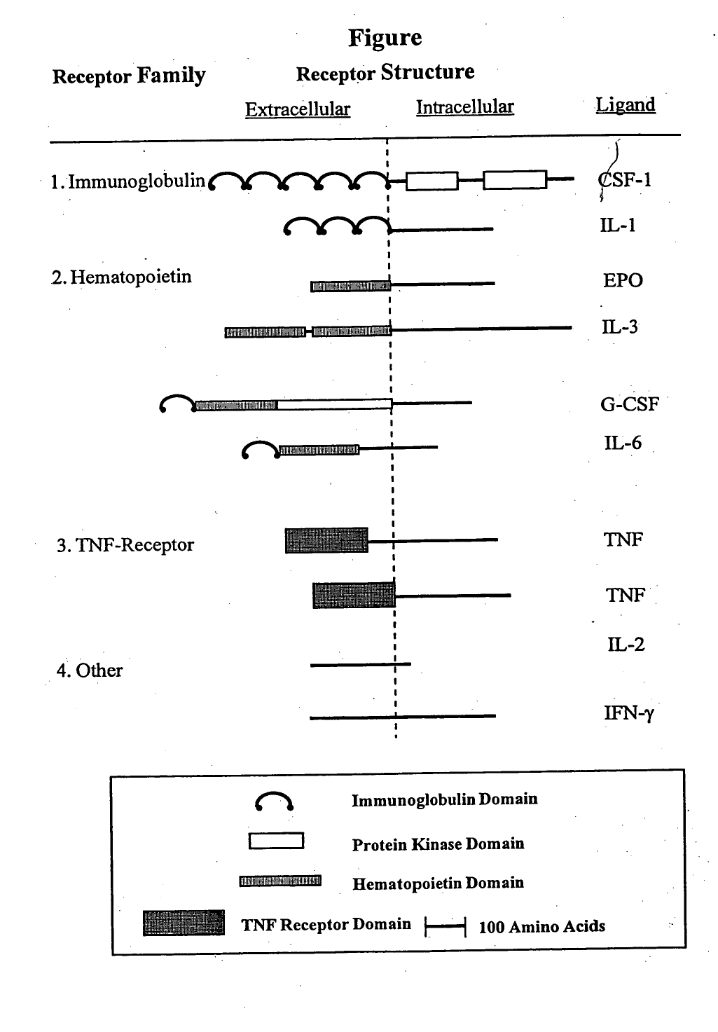

### BACKGROUND OF THE INVENTION

Proliferation and differentiation of cells of multicellular organisms are controlled by hormones and polypeptide growth factors. These diffusable molecules allow cells to communicate with each other and act in concert to form cells and organs, and to repair and regenerate damaged tissue. Examples of hormones and growth factors include the steroid hormones (e.g. estrogen, testosterone), parathyroid hormone, follicle stimulating hormone, the interleukins, platelet derived growth factor (PDGF), epidermal growth factor (EGF), granulocyte-macrophage colony stimulating factor (GM-CSF), erythropoietin (EPO) and calcitonin.

Hormones and growth factors influence cellular metabolism by binding to receptors. Receptors may be integral membrane proteins that are linked to signalling pathways within the cell, such as second messenger systems. Other classes of receptors are soluble molecules, such as the transcription factors.

Of particular interest are receptors for cytokines, molecules that promote the proliferation and/or differentiation of cells. Examples of cytokines include erythropoietin (EPO), which stimulates the development of red blood cells; thrombopoietin (TPO), which stimulates development of cells of the megakaryocyte lineage; and granulocyte-colony stimulating factor (G-CSF), which stimulates development of neutrophils. These cytokines are useful in restoring normal blood cell levels in patients suffering from anemia or receiving chemotherapy for cancer. The demonstrated in vivo activities of these cytokines illustrates the enormous clinical potential of, and need for, other cytokines, cytokine agonists, and cytokine antagonists. The present invention addresses this need by providing novel cytokine receptors and related compositions and methods.

### SUMMARY OF THE INVENTION

Within one aspect, the present invention provides an isolated polynucleotide encoding a ligand-binding receptor polypeptide. The polypeptide comprises a sequence of amino acids selected from the group consisting of (a) residues 141 to 337 of SEQ ID NO:2; (b) allelic variants of (a); and (c) sequences that are at least 80% identical to (a) or (b). Within one embodiment, the polypeptide comprises residues 141 to 337 of SEQ ID NO:2 or SEQ ID NO:4. Within another embodiment, the polypeptide encoded by the isolated polynucleotide further comprises a transmembrane domain. The transmembrane domain may comprise residues 340 to 363 of SEQ ID NO:2, or an allelic variant thereof. Within another embodiment, the polypeptide encoded by the isolated polynucleotide further comprises an intracellular domain, such as an intracellular domain comprising residues 364 to 380 of SEQ ID NO:2, or an allelic variant thereof. Within further embodiments, the polynucleotide encodes a polypeptide that comprises residues 25 to 337, 1 to 337, or 1 to 380 of SEQ ID NO:2 or SEQ ID NO:4. Within an additional embodiment, the polypeptide further comprises an affinity tag. Within a further embodiment, the polynucleotide is DNA.

Within a second aspect of the invention there is provided an expression vector comprising (a) a transcription promoter; (b) a DNA segment encoding a secretory peptide and a ligand-binding receptor polypeptide, wherein the polypeptide comprises a sequence of amino acids selected from the group consisting of: (i) residues 141 to 337 of SEQ ID NO:2; (ii) allelic variants of (i); and (iii) sequences that are at least 80% identical to (i) or (ii); and (c) a transcription terminator, wherein the promoter, DNA segment, and terminator are operably linked. The ligand-binding receptor polypeptide may further comprise a transmembrane domain, or a transmembrane domain and an intracellular domain.

Within a third aspect of the invention there is provided a cultured eukaryotic cell into which has been introduced an expression vector as disclosed above, wherein said cell expresses a receptor polypeptide encoded by the DNA segment. Within one embodiment, the cell further expresses a signalling subunit, such as a hematopoietic receptor β_{c} subunit. Within another embodiment, the cell is dependent upon an exogenously supplied hematopoietic growth factor for proliferation.

Within a fourth aspect of the invention there is provided an isolated polypeptide comprising a segment selected from the group consisting of (a) residues 141 to 337 of SEQ ID NO:2; (b) allelic variants of (a); and (c) sequences that are at least 80% identical to (a) or (b), wherein said polypeptide is substantially free of transmembrane and intracellular domains ordinarily associated with hematopoietic receptors. Within one embodiment, the polypeptide further comprises an immunoglobulin F_{c} polypeptide. Within a another embodiment, the polypeptide further comprises an affinity tag, such as polyhistidine, protein A, glutathione S transferase, or an immunoglobulin heavy chain constant region. Within a further embodiment, the polypeptide comprises residues 25-337 of SEQ ID NO:2, an allelic variant of SEQ ID.NO:2, or a sequence that is at least 80% identical to residues 25-337 of SEQ ID NO:2 or an allelic variant of SEQ ID NO:2.

Within a further aspect of the invention there is provided a chimeric polypeptide consisting essentially of a first portion and a second portion joined by a peptide bond. The first portion of the chimeric polypeptide consists essentially of a ligand binding domain of a receptor polypeptide selected from the group consisting of (a) a receptor polypeptide as shown in SEQ ID NO:2; (b) allelic variants of SEQ ID NO:2; and (c) receptor polypeptides that are at least 80% identical to (a) or (b). The second portion of the chimeric polypeptide consists essentially of an affinity tag. Within one embodiment the affinity tag is an immunoglobulin F_{c} polypeptide. The invention also provides expression vectors encoding the chimeric polypeptides and host cells transfected to produce the chimeric polypeptides.

The invention also provides a method for detecting a ligand within a test sample, comprising contacting a test sample with a polypeptide as disclosed above, and detecting binding of the polypeptide to ligand in the sample. Within one embodiment the polypeptide further comprises transmembrane and intracellular domains. The polypeptide can be membrane bound within a cultured cell, wherein the detecting step comprises measuring a biological response in the cultured cell. Within another embodiment, the polypeptide is immobilized on a solid support.

Within an additional aspect of the invention there is provided an antibody that specifically binds to a polypeptide as disclosed above.

These and other aspects of the invention will become evident upon reference to the following detailed description and the attached drawing.

### BRIEF DESCRIPTION OF THE DRAWING

The FIGURE illustrates conserved structural features in cytokine receptors.

### DETAILED DESCRIPTION OF THE INVENTION

The term “allelic variant” is used herein to denote any of two or more alternative forms of a gene occupying the same chromosomal locus. Allelic variation arises naturally through mutation, and may result in phenotypic polymorphism within populations. Gene mutations can be silent (no change in the encoded polypeptide) or may encode polypeptides having altered amino acid sequence. The term allelic variant is also used herein to denote a protein encoded by an allelic variant of a gene.

The term “expression vector” is used to denote a DNA molecule, linear or circular, that comprises a segment encoding a polypeptide of interest operably linked to additional segments that provide for its transcription. Such additional segments include promoter and terminator sequences, and may also include one or more origins of replication, one or more selectable markers, an enhancer, a polyadenylation signal, etc. Expression vectors are generally derived from plasmid or viral DNA, or may contain elements of both.

The term “isolated”, when applied to a polynucleotide, denotes that the polynucleotide has been removed from its natural genetic milieu and is thus free of other extraneous or unwanted coding sequences, and is in a form suitable for use within genetically engineered protein production systems.

“Operably linked”, when referring to DNA segments, indicates that the segments are arranged so that they function in concert for their intended purposes, e.g. transcription initiates in the promoter and proceeds through the coding segment to the terminator.

A “polynucleotide” is a single- or double-stranded polymer of deoxyribonucleotide or ribonucleotide bases read from the 5′ to the 3′ end. Polynucleotides include RNA and DNA, and may be isolated from natural sources, synthesized in vitro, or prepared from a combination of natural and synthetic molecules.

The term “promoter” is used herein for its art-recognized meaning to denote a portion of a gene containing DNA sequences that provide for the binding of RNA polymerase and initiation of transcription. Promoter sequences are commonly, but not always, found in the 5′ non-coding regions of genes.

The term “receptor” is used herein to denote a cell-associated protein, or a polypeptide subunit of such a protein, that binds to a bioactive molecule (the “ligand”) and mediates the effect of the ligand on the cell. Binding of ligand to receptor results in a conformational change in the receptor. (and, in some cases, receptor multimerization, i.e., association of identical or different receptor subunits) that causes interactions between the effector domain(s) and other molecule(s) in the cell. These interactions in turn lead to alterations in the metabolism of the cell. Metabolic events that are linked to receptor-ligand interactions include gene transcription, phosphorylation, dephosphorylation, cell proliferation, increases in cyclic AMP production, mobilization of cellular calcium, mobilization of membrane lipids, cell adhesion, hydrolysis of inositol lipids and hydrolysis of phospholipids. The term “receptor polypeptide” is used to denote complete receptor polypeptide chains and portions thereof, including isolated functional domains (e.g., ligand-binding domains).

A “secretory signal sequence” is a DNA sequence that encodes a polypeptide (a “secretory peptide”) that, as a component of a larger polypeptide, directs the larger polypeptide through a secretory pathway of a cell in which it is synthesized. The larger polypeptide is commonly cleaved to remove the secretory peptide during transit through the secretory pathway.

A “soluble receptor” is a receptor polypeptide that is not bound to a cell membrane. Soluble receptors are most commonly ligand-binding receptor polypeptides that lack transmembrane and cytoplasmic domains. Soluble receptors can comprise additional amino acid residues, such as affinity tags that provide for purification of the polypeptide or provide sites for attachment of the polypeptide to a substrate, or immunoglobulin constant region sequences. Many cell-surface receptors have naturally occurring, soluble counterparts that are produced by proteolysis or translated from alternatively spliced mRNAs. Receptor polypeptides are said to be substantially free of transmembrane and intracellular polypeptide segments when they lack sufficient portions of these segments to provide membrane anchoring or signal transduction, respectively.

The present invention is based in part upon the discovery of a novel DNA sequence that encodes a protein having the structure of a cytokine receptor, including the conserved WSXWS motif (SEQ ID NO:5). Analysis of the tissue distribution of the mRNA corresponding to this novel DNA showed that it was highly expressed in the testes, suggesting that the receptor mediates processes of progenitor cell growth and development, such as spermatogenesis. The receptor is also expressed at lower levels in pituitary. Subsequently, the receptor was shown to bind interleukin 13 (IL-13). The human cDNA was subsequently used to clone the orthologous receptor from Celebus macaque. The receptor has been designated “ZCytor2”.

Cytokine receptors subunits are characterized by a multi-domain structure comprising a ligand-binding domain and an effector domain that is typically involved in signal transduction. Multimeric cytokine receptors include homodimers (e.g., PDGF receptor αα and ββ isoforms, erythropoietin receptor, MPL [thrombopoietin receptor], and G-CSF receptor), heterodimers whose subunits each have ligand-binding and effector domains (e.g., PDGF receptor αβ isoform), and multimers having component subunits with disparate functions (e.g., IL-2, IL-3, IL-4, IL-5, IL-6, IL-7, and GM-CSF receptors). Some receptor subunits are common to a plurality of receptors. For example; the AIC2B subunit, which cannot bind ligand on its own but includes an intracellular signal transduction domain, is a component of IL-3 and GM-CSF receptors. Many cytokine receptors can be placed into one of four related families on the basis of their structures (see Figure) and functions. Hematopoietic receptors, for example, are characterized by the presence of a domain containing conserved cysteine residues and the WSXWS motif (SEQ ID NO:5). Additional domains, including protein kinase domains; fibronectin type III domains; and immunoglobulin domains, which are characterized by disulfide-bonded loops, are present in certain hematopoietic receptors. Cytokine receptor structure has been reviewed by Urdal, Ann. Resorts Med. Chem. 26:221-228, 1991 and Cosman, Cytokine 5:95-106, 1993. It is generally believed that under selective pressure for organisms to acquire new biological functions, new receptor family members arose from duplication of existing receptor genes leading to the existence of multi-gene families. Family members thus contain vestiges of the ancestral gene, and these characteristic features can be exploited in the isolation and identification of additional family members. The cytokine receptor superfamily is subdivided as shown in Table 1.

TABLE 1
Cytokine Receptor Superfamily

Immunoglobulin family
CSF-1 receptor
MGF receptor
IL-1 receptor
PDGF receptor
Hematopoietin family
erythropoietin receptor
G-CSF receptor
IL-2 receptor β-subunit
IL-3 receptor
IL-4 receptor
IL-5 receptor
IL-6 receptor
IL-7 receptor
IL-9 receptor
GM-CSF receptor α-subunit
GM-CSF receptor β-subunit
Prolactin receptor
CNTF receptor
Oncostatin M receptor
Leukemia inhibitory factor receptor
Growth hormone receptor
MPL
Leptin receptor
TNF receptor family
TNF (p80) receptor
TNF (p60) receptor
TNFR-RP
CD27
CD30
CD40
4-1BB
OX-40
Fas
NGF receptor
Other
IL-2 receptor α-subunit
IL-15 receptor α-subunit
IFN-γ receptor

Cell-surface cytokine receptors are further characterized by the presence of additional domains. These receptors are anchored in the cell membrane by a transmembrane domain characterized by a sequence of hydrophobic amino acid residues (typically about 21-25 residues), which is commonly flanked by positively charged residues (Lys or Arg). On the opposite end of the protein from the extracellular domain and separated from it by the transmembrane domain is an intracellular domain.

The novel receptor of the present invention was initially identified by the presence of the conserved WSXWS motif (SEQ ID NO:5). Analysis of a human cDNA clone encoding ZCytor2 (SEQ ID NO:1) revealed an open reading frame encoding 380 amino acids (SEQ ID NO:2) comprising an extracellular ligand-binding domain of approximately 315 amino acid residues (residues 25-339 of SEQ ID NO:2), a transmembrane domain of approximately 24 amino acid residues (residues 340-363 of SEQ ID NO:2), and a short intracellular domain of approximately 17 amino acid residues (residues 364-380 of SEQ ID NO:2). Those skilled in the art will recognize that these domain boundaries are approximate and are based on alignments with known proteins and predictions of protein folding. Deletion of residues from the ends of the domains is possible. For example, the core ligand binding region is believed to reside within residues 141-337 of SEQ ID NO:2. Structural analysis indicates that the polypeptide regions from Cys145 through Cys155 and from Cys184 through Cys197 of SEQ ID NO:2 are cysteine loops that are important ligand-binding sites. Relatively small, ligand-binding receptor polypeptides are thus provided by the present invention.

The deduced amino acid sequence of Zcytor2 indicates that it belongs to the same subfamily as the IL-3, IL-5 and GM-CSF receptor a subunits. These a receptor subunits are ligand-specific proteins that combine with a common signalling subunit (β-subunit) to form a signalling complex in the presence of the cognate ligand. The β-subunit for this receptor subfamily has been previously identified in mouse (Itoh et al., Science 247:324-327, 1989; Gorman et al., Proc. Natl. Acad. Sci. USA 87:5459-5463, 1990) and human (Hayashida, et al., Proc. Natl. Aca. Sci. USA 87:9655-9659, 1990). The mouse β-subunit occurs in two isoforms, denoted AIC2A and AIC2B, whereas in human only one form (denoted β_{c}) has been identified. β_{c} is also a member of the hematopoietin receptor family in that it contains a WSXWS motif (SEQ ID NO:5) and a single transmembrane domain. β_{c} also contains a sizable intracellular domain capable of interacting with cytoplasmic proteins for signal propagation. In the alternative, Zcytor2 may combine with one or more of gp130 (Hibi et al., Cell 63:1149-1157, 1990), the IL-4 α-subunit (Idzerda, et al., J. Exp. Med. 171:861, 1990), or the IL-13. α-subunit (Hilton et al., Proc. Natl. Acad. Sci. USA 93:497-501, 1996) in a tissue specific manner to form dimeric or trimeric complexes. Binding data for Zcytor2 suggest that this receptor subunit may form an IL-13 receptor complex in testes and pituitary that is different from the immune system IL-13 receptor.

Within preferred embodiments of the invention the isolated polynucleotides will hybridize to similar sized regions of SEQ ID NO:1, SEQ ID NO:3, or SEQ ID NO:6, or a sequence complementary thereto, under stringent conditions. In general, stringent conditions are selected to be about 5° C. lower than the thermal melting point (Tₘ) for the specific sequence at a defined ionic strength and pH. The Tₘ is the temperature (under defined ionic strength and pH) at which 50% of the target sequence hybridizes to a perfectly matched probe. Typical stringent conditions are those in which the salt concentration is at least about 0.02 M at pH 7 and the temperature is at least about 60° C. As previously noted, the isolated polynucleotides of the present invention include DNA and RNA. Methods for isolating DNA and RNA are well known in the art. It is generally preferred to isolate RNA from testis, including whole testis tissue extracts or testicular cells, such as Sertoli cells, Leydig cells, spermatogonia, or epididymis, although. DNA can also be prepared using RNA from other tissues or isolated as genomic DNA. Total RNA can be prepared using guanidine HCl extraction followed by isolation by centrifugation in a CsCl gradient (Chirgwin et al., Biochemistry 18:52-94, 1979). Poly (A)+ RNA is prepared from total RNA using the method of Aviv and Leder (Proc. Natl. Acad. Sci. USA 69:1408-1412, 1972). Complementary DNA (cDNA) is prepared from poly(A)+ RNA using known methods. Polynucleotides encoding Zcytor2 polypeptides are then identified and isolated by, for example, hybridization or PCR.

Those skilled in the art will recognize that the sequences disclosed in SEQ ID NOS:1, 2, 6, and 7 represent single alleles of the human and macaque ZCytor2 receptors, respectively. Allelic variants of these sequences can be cloned by probing cDNA or genomic libraries from different individuals according to standard procedures. DNA and protein sequences from an additional human clone are shown in SEQ ID NOS: 3 and 4.

The present invention further provides counterpart receptors and polynucleotides from other species (“species orthologs”) of particular interest are ZCytor2 receptors from other mammalian species, including murine, porcine, ovine, bovine, canine, feline, equine, and other primate receptors. Species orthologs of the human and macaque ZCytor2 receptors can be cloned using information and compositions provided by the present invention in combination with conventional cloning techniques. For example, a cDNA can be cloned using mRNA obtained from a tissue or cell type that expresses the receptor. Suitable sources of mRNA can be identified by probing Northern blots with probes designed from the sequences disclosed herein. A library is then prepared from mRNA of a positive tissue or cell line. A receptor-encoding cDNA can then be isolated by a variety of methods, such as by probing with a complete or partial human or macaque cDNA or with one or more sets of degenerate probes based on the disclosed sequences. A cDNA can also be cloned using the polymerase chain reaction, or PCR (Mullis, U.S. Pat. No. 4,683,202), using primers designed from the sequences disclosed herein. Within an additional method, the cDNA library can be used to transform or transfect host cells, and expression of the cDNA of interest can be detected with an antibody to the receptor. Similar techniques can also be applied to the isolation of genomic clones.

The present invention also provides isolated receptor polypeptides that are substantially homologous to the receptor polypeptides of SEQ ID NO: 2 or SEQ ID NO:7 and their species orthologs. By “isolated” is meant a protein or polypeptide that is found in a condition other than its native environment, such as apart from blood and animal tissue. In a preferred form, the isolated polypeptide is substantially free of other polypeptides, particularly other polypeptides of animal origin. It is prefered to provide the polypeptides in a highly purified form, i.e. greater than 95% pure, more preferably greater than 99% pure. The term “substantially homologous” is used herein to denote polypeptides having 50%, preferably 60%, more preferably at least 80%, sequence identity to the sequences shown in SEQ ID NO:2, 4, or 7 or their species orthologs. Such polypeptides will more preferably be at least 90% identical, and most preferably 0.95% or more identical to SEQ ID NO:2, 4 or 7 or their species orthologs. Percent sequence identity is determined by conventional methods. See, for example, Altschul et al., Bull. Math. Bio. 48: 603-616, 1986 and Henikoff and Henikoff, Proc. Natl. Acad. Sci. USA 89:10915-10919, 1992. Briefly, two amino acid sequences are aligned to optimize the alignment scores using a gap opening penalty of 10, a gap extension penalty of 1, and the “blosum 62” scoring matrix of Henikoff and Henikoff (ibid.) as shown in Table 2 (amino acids are indicated by the standard one-letter codes). The percent identity is then calculated as:

$$\frac{Total number of identical matches}{\begin{matrix}[ length of the longer sequence plus the \\ number of gaps introduced into the longer \\ sequence in order to align the two sequences ]\end{matrix}} \times 100$$

TABLE 2

| | A | R | N | D | C | Q | E | G | H | I | L | K | M | F | P | S | T | W | Y | V |
|---|---|---|---|---|---|---|---|---|---|---|---|---|---|---|---|---|---|---|---|---|
| A | 4 | | | | | | | | | | | | | | | | | | | |
| R | −1 | 5 | | | | | | | | | | | | | | | | | | |
| N | −2 | 0 | 6 | | | | | | | | | | | | | | | | | |
| D | −2 | −2 | 1 | 6 | | | | | | | | | | | | | | | | |
| C | 0 | −3 | −3 | −3 | 9 | | | | | | | | | | | | | | | |
| Q | −1 | 1 | 0 | 0 | −3 | 5 | | | | | | | | | | | | | | |
| E | −1 | 0 | 0 | 2 | −4 | 2 | 5 | | | | | | | | | | | | | |
| G | 0 | −2 | 0 | −1 | −3 | −2 | −2 | 6 | | | | | | | | | | | | |
| H | −2 | 0 | 1 | −1 | −3 | 0 | 0 | −2 | 8 | | | | | | | | | | | |
| I | −1 | −3 | −3 | −3 | −1 | −3 | −3 | −4 | −3 | 4 | | | | | | | | | | |
| L | −1 | −2 | −3 | −4 | −1 | −2 | −3 | −4 | −3 | 2 | 4 | | | | | | | | | |
| K | −1 | 2 | 0 | −1 | −3 | 1 | 1 | −2 | −1 | −3 | −2 | 5 | | | | | | | | |
| M | −1 | −1 | −2 | −3 | −1 | 0 | −2 | −3 | −2 | 1 | 2 | −1 | 5 | | | | | | | |
| F | −2 | −3 | −3 | −3 | −2 | −3 | −3 | −3 | −1 | 0 | 0 | −3 | 0 | 6 | | | | | | |
| P | −1 | −2 | −2 | −1 | −3 | −1 | −1 | −2 | −2 | −3 | −3 | −1 | −2 | −4 | 7 | | | | | |
| S | 1 | −1 | 1 | 0 | −1 | 0 | 0 | 0 | −1 | −2 | −2 | 0 | −1 | −2 | −1 | 4 | | | | |
| T | 0 | −1 | 0 | −1 | −1 | −1 | −1 | −2 | −2 | −1 | −1 | −1 | −1 | −2 | −1 | 1 | 5 | | | |
| W | −3 | −3 | −4 | −4 | −2 | −2 | −3 | −2 | −2 | −3 | −2 | −3 | −1 | 1 | −4 | −3 | −2 | 11 | | |
| Y | −2 | −2 | −2 | −3 | −2 | −1 | −2 | −3 | 2 | −1 | −1 | −2 | −1 | 3 | −3 | −2 | −2 | 2 | 7 | |
| V | 0 | −3 | −3 | −3 | −1 | −2 | −2 | −3 | −3 | 3 | 1 | −2 | 1 | −1 | −2 | −2 | 0 | −3 | −1 | 4 |

Sequence identity of polynucleotide molecules is determined by similar methods using a ratio as disclosed above.

Substantially homologous proteins and polypeptides are characterized as having one or more amino acid substitutions, deletions or additions. These changes are preferably of a minor nature, that is conservative amino acid substitutions (see Table 3) and other substitutions that do not significantly affect the folding or activity of the protein or polypeptide; small deletions, typically of one to about 30 amino acids; and small amino- or carboxyl-terminal extensions, such as an amino-terminal methionine residue, a small linker peptide of up to about 20-25 residues, or a small extension that facilitates purification (an affinity tag), such as a poly-histidine tract, protein A (Nilsson et al., EMBO J. 4:1075, 1985; Nilsson et al., Methods Enzymol. 198:3, 1991), glutathione S transferase (Smith and Johnson, Gene 67:31, 1988), or other antigenic epitope or binding domain. See, in general Ford et al., Protein Expression and Purification 2: 95-107, 1991, which is incorporated herein by reference. DNAs encoding affinity tags are available from commercial suppliers (e.g., Pharmacia Biotech, Piscataway, N.J.).

TABLE 3
Conservative amino acid substitutions

| | |
|---|---|
| Basic: | arginine |
| | lysine |
| | histidine |
| Acidic: | glutamic acid |
| | aspartic acid |
| Polar: | glutamine |
| | asparagine |
| Hydrophobic: | leucine |
| | isoleucine |
| | valine |
| Aromatic: | phenylalanine |
| | tryptophan |
| | tyrosine |
| Small: | glycine |
| | alanine |
| | serine |
| | threonine |
| | methionine |

Essential amino acids in the receptor polypeptides of the present invention can be identified according to procedures known in the art, such as site-directed mutagenesis or alanine-scanning mutagenesis (Cunningham and Wells, Science 244, 1081-1085, 1989; Bass et al., Proc. Natl. Acad. Sci. USA 88:4498-4502, 1991). In the latter technique, single alanine mutations are introduced at every residue in the molecule, and the resultant mutant molecules are tested for biological activity (e.g., ligand binding and signal transduction) to identify amino acid residues that are critical to the activity of the molecule. Sites of ligand-receptor interaction can also be determined by analysis of crystal structure as determined by such techniques as nuclear magnetic resonance, crystallography or photoaffinity labeling. See, for example, de Vos et al., Science 255:306-312, 1992; Smith et al., J. Mol. Biol. 224:899-904, 1992; Wlodaver et al., FEBS Lett. 309:59-64, 1992. The identities of essential amino acids can also be inferred from analysis of homologies with related receptors.

Multiple amino acid substitutions can be made and tested using known methods of mutagenesis and screening, such as those disclosed by Reidhaar-Olson and Sauer (Science 241:53-57, 1988) or Bowie and Sauer (Proc. Natl. Acad. Sci. USA 86:2152-2156, 1989). Briefly, these authors disclose methods for simultaneously randomizing two or more positions in a polypeptide, selecting for functional polypeptide, and then sequencing the mutagenized polypeptides to determine the spectrum of allowable substitutions at each position. Other methods that can be used include phage display (e.g., Lowman et al., Biochem. 30:10832-10837, 1991; Ladner et al., U.S. Pat. No. 5,223,409; Huse, WIPO Publication WO 92/06204) and region-directed mutagenesis (Derbyshire et al., Gene 46:145, 1986; Ner et al., DNA 7:127, 1988)

Mutagenesis methods as disclosed above can be combined with high-throughput screening methods to detect activity of cloned, mutagenized receptors in host cells. Preferred assays in this regard include cell proliferation assays and biosensor-based ligand-binding assays, which are described below. Mutagenized DNA molecules that encode active receptors or portions thereof (e.g., ligand-binding fragments) can be recovered from the host cells and rapidly sequenced using modern equipment. These methods allow the rapid determination of the importance of individual amino acid residues in a polypeptide of interest, and can be applied to polypeptides of unknown structure.

Using the methods discussed above, one of ordinary skill in the art can prepare a variety of polypeptides that are substantially homologous to residues 141 to 337 of SEQ ID NO:2 or allelic variants thereof and retain the ligand-binding properties of the wild-type receptor. Such polypeptides may include additional amino acids from an extracellular ligand-binding domain of a Zcytor2 receptor as well as part or all of the transmembrane and intracellular domains. Such polypeptides may also include additional polypeptide segments as generally disclosed above.

The receptor polypeptides of the present invention, including full-length receptors, receptor fragments (e.g. ligand-binding fragments), and fusion polypeptides can be produced in genetically engineered host cells according to conventional techniques. Suitable host cells are those cell types that can be transformed or transfected with exogenous DNA and grown in culture, and include bacteria, fungal cells, and cultured higher eukaryotic cells. Eukaryotic cells, particularly cultured cells of multicellular organisms, are preferred. Techniques for manipulating cloned DNA molecules and introducing exogenous DNA into a variety of host cells are disclosed by Sambrook et al., Molecular Cloning: A Laboratory Manual, 2nd ed., Cold Spring Harbor Laboratory Press, Cold Spring Harbor, N.Y., 1989, and Ausubel et al., ibid., which are incorporated herein by reference.

In general, a DNA sequence encoding a ZCytor2 receptor polypeptide is operably linked to other genetic elements required for its expression, generally including a transcription promoter and terminator, within an expression vector. The vector will also commonly contain one or more selectable markers and one or more origins of replication, although those skilled in the art will recognize that within certain systems selectable markers may be provided on separate vectors, and replication of the exogenous DNA may be provided by integration into the host cell genome. Selection of promoters, terminators, selectable markers, vectors and other elements is a matter of routine design within the level of ordinary skill in the art. Many such elements are described in the literature and are available through commercial suppliers.

To direct a ZCytor2 receptor polypeptide into the secretory pathway of a host cell, a secretory signal sequence (also known as a leader sequence, prepro sequence or pre sequence) is provided in the expression vector. The secretory signal sequence may be that of the receptor, or may be derived from another secreted protein (e.g., t-PA) or synthesized de novo. The secretory signal sequence is joined to the ZCytor2 DNA sequence in the correct reading frame. Secretory signal sequences are commonly positioned 5′ to the DNA sequence encoding the polypeptide of interest, although certain signal sequences may be positioned elsewhere in the DNA sequence of interest (see, e.g., Welch et al., U.S. Pat. No. 5,037,743; Holland et al., U.S. Pat. No. 5,143,830).

Cultured mammalian cells are preferred hosts within the present invention. Methods for introducing exogenous DNA into mammalian host cells include calcium phosphate-mediated transfection (Wigler et al., Cell 14:725, 1978; Corsaro and Pearson, Somatic Cell Genetics 7:603, 1981: Graham and Van der Eb, Virology 52:456, 1973), electroporation (Neumann et al., EMBO J. 1:841-845, 1982), DEAE-dextran mediated transfection (Ausubel et al., eds., Current Protocols in Molecular Biology, John Wiley and Sons, Inc., NY, 1987), and liposome-mediated transfection (Hawley-Nelson et al., Focus 15:73, 1993; Ciccarone et al., Focus 15:80, 1993), which are incorporated herein by reference. The production of recombinant polypeptides in cultured mammalian cells is disclosed, for example, by Levinson et al., U.S. Pat. No. 4,713,339; Hagen et al., U.S. Pat. No. 4,784,950; Palmiter et al., U.S. Pat. No. 4,579,821; and Ringold, U.S. Pat. No. 4,656,134, which are incorporated herein by reference. Suitable cultured mammalian cells include the COS-1 (ATCC No. CRL 1650), COS-7 (ATCC No. CRL 1651), BHK (ATCC No. CRL 1632), BHK 570 (ATCC No. CRL 10314), 293 (ATCC No. CRL 1573; Graham et al., J. Gen. Virol. 36:59-72, 1977) and Chinese hamster ovary (e.g. CHO-K1; ATCC No. CCL 61) cell lines. Additional suitable cell lines are known in the art and available from public depositories such as the American Type Culture Collection, Rockville, Md. In general, strong transcription promoters are preferred, such as promoters from SV-40 or cytomegalovirus. See, e.g., U.S. Pat. No. 4,956,288. Other suitable promoters include those from metallothionein genes (U.S. Pat. Nos. 4,579,821 and 4,601,978, which are incorporated herein by reference) and the adenovirus major late promoter.

Drug selection is generally used to select for cultured mammalian cells into which foreign DNA has been inserted. Such cells are commonly referred to as “transfectants”. Cells that have been cultured in the presence of the selective agent and are able to pass the gene of interest to their progeny are referred to as “stable transfectants.” A preferred selectable marker is a gene encoding resistance to the antibiotic neomycin. Selection is carried out in the presence of a neomycin-type drug, such as G-418 or the like. Selection systems may also be used to increase the expression level of the gene of interest, a process referred to as “amplification.” Amplification is carried out by culturing transfectants in the presence of a low level of the selective agent and then increasing the amount of selective agent to select for cells that produce high levels of the products of the introduced genes. A preferred amplifiable selectable marker is dihydrofolate reductase, which confers resistance to methotrexate. Other drug resistance genes (e.g. hygromycin resistance, multi-drug resistance, puromycin acetyltransferase) can also be used.

Other higher eukaryotic cells can also be used as hosts, including insect cells, plant cells and avian cells. Transformation of insect cells and production of foreign polypeptides therein is disclosed by Guarino et al., U.S. Pat. No. 5,162,222; Bang et al., U.S. Pat. No. 4,775,624; and WIPO publication WO 94/06463, which are incorporated herein by reference. The use of Agrobacterium rhizogenes as a vector for expressing genes in plant cells has been reviewed by Sinkar et al., J. Biosci. (Bangalore) 11:47-58, 1987.

Fungal cells, including yeast cells, and particularly cells of the genus Saccharomyces, can also be used within the present invention, such as for producing receptor fragments or polypeptide fusions. Methods for transforming yeast cells with exogenous DNA and producing recombinant polypeptides therefrom are disclosed by, for example, Kawasaki, U.S. Pat. No. 4,599,311; Kawasaki et al., U.S. Pat. No. 4,931,373; Brake., U.S. Pat. No. 4,870,008; Welch et al., U.S. Pat. No. 5,037,743; and Murray et al., U.S. Pat. No. 4,845,075, which are incorporated herein by reference. Transformed cells are selected by phenotype determined by the selectable marker, commonly drug resistance or the ability to grow in the absence of a particular nutrient (e.g., leucine). A preferred vector system for use in yeast is the POT1 vector system disclosed by Kawasaki et al. (U.S. Pat. No. 4,931,373), which allows transformed cells to be selected by growth in glucose-containing media. Suitable promoters and terminators for use in yeast include those from glycolytic enzyme genes (see, e.g., Kawasaki, U.S. Pat. No. 4,599,311; Kingsman et al., U.S. Pat. No. 4,615,974; and Bitter, U.S. Pat. No. 4,977,092, which are incorporated herein by reference) and alcohol dehydrogenase genes. See also U.S. Pat. Nos. 4,990,446; 5,063,154; 5,139,936 and 4,661,454, which are incorporated herein by reference. Transformation systems for other yeasts, including Hansenula polymorpha, Schizosaccharomyces pombe, Kluyveromyces lactis, Kluyveromyces fragilis, Ustilago maydis, Pichia pastoris, Pichia methanolica, Pichia guillermondii and Candida maltosa are known in the art. See, for example, Gleeson et al., J. Gen. Microbiol. 132:3459-3465, 1986 and Cregg, U.S. Pat. No. 4,882,279. Aspergillus cells may be utilized according to the methods of McKnight et ale, U.S. Pat. No. 4,935,349, which is incorporated herein by reference. Methods for transforming Acremonium chrysogenum are disclosed by Sumino et al., U.S. Pat. No. 5,162,228, which is incorporated herein by reference. Methods for transforming Neurospora are disclosed by Lambowitz, U.S. Pat. No. 4,486,533, which is incorporated herein by reference.

Transformed or transfected host cells are cultured according to conventional procedures in a culture medium containing nutrients and other components required for the growth of the chosen host cells. A variety of suitable media, including defined media and complex media, are known in the art and generally include a carbon source, a nitrogen source, essential amino acids, vitamins and minerals. Media may also contain such components as growth factors or serum, as required. The growth medium will generally select for cells containing the exogenously added DNA by, for example, drug selection or deficiency in an essential nutrient which is complemented by the selectable marker carried on the expression vector or co-transfected into the host cell.

Within one aspect of the present invention, a novel receptor is produced by a cultured cell, and the cell is used to screen for ligands for the receptor, including the natural ligand, as well as agonists and antagonists of the natural ligand. To summarize this approach, a cDNA or gene encoding the receptor is combined with other genetic elements required for its expression (e.g., a transcription promoter), and the resulting expression vector is inserted into a host cell. Cells that express the DNA and produce functional receptor are selected and used within a variety of screening systems.

Mammalian cells suitable for use in expressing ZCytor2 receptors and transducing a receptor-mediated signal include cells that express a β-subunit, such as the human β_{c} subunit. In this regard it is generally preferred to employ a cell that is responsive to other cytokines that bind to receptors in the same subfamily, such as IL-3 or GM-CSF, because such cells will contain the requisite signal transduction pathway(s). It is also preferred to use a cell from the same species as the receptor to be expressed. Within a preferred embodiment, the cell is dependent upon an exogenously supplied hematopoietic growth factor for its proliferation. Preferred cell lines of this type are the human TF-1 cell line (ATCC number CRL-2003) and the AML-193 cell line (ATCC number CRL-9589), which are GM-CSF-dependent human leukemic cell lines. In the alternative, suitable host cells can be engineered to produce a β-subunit (e.g., β_{c}) or other cellular component needed for the desired cellular response. For example, the murine cell line BaF3 (Palacios and Steinmetz, Cell 41: 727-734, 1985; Mathey-Prevot et al., Mol. Cell. Biol. 6: 4133-4135, 1986) or a baby hamster kidney (BHK) cell line can be transfected to express the human β_{c} subunit (also known as KH97) as well as a ZCytor2 receptor. The latter approach is advantageous because cell lines can be engineered to express receptor subunits from any species, thereby overcoming potential limitations arising from species specificity. In the alternative, species orthologs of the human receptor cDNA can be cloned and used within cell lines from the same species, such as a mouse cDNA in the BaF3 cell line. Cell lines that are dependent upon one hematopoietic growth factor, such as GM-CSF, can thus be engineered to become dependent upon a Zcytor2 ligand.

Cells expressing functional receptor are used within screening assays. A variety of suitable assays are known in the art. These assays are based on the detection of a biological response in a target cell. One such assay is a cell proliferation assay. Cells are cultured in the presence or absence of a test compound, and cell proliferation is detected by, for example, measuring incorporation of tritiated thymidine or by calorimetric assay based on the metabolic breakdown of 3-(4,5-dimethylthiazol-2-yl)-2,5-diphenyl tetrazolium bromide (MTT) (Mosman, J. Immunol. Meth. 65: 55-63, 1983). An alternative assay format uses cells that are further engineered to express a reporter gene. The reporter gene is linked to a promoter element that is responsive to the receptor-linked pathway, and the assay detects activation of transcription of the reporter gene. A preferred promoter element in this regard is a serum response element, or SRE (see, e.g., Shaw et al., Cell 56:563-572, 1989). A preferred such reporter gene is a luciferase gene (de Wet et al., Mol. Cell. Biol. 7:725, 1987). Expression of the luciferase gene is detected by luminescence using methods known in the art (e.g., Baumgartner et al., J. Biol. Chem. 269:29094-29101, 1994; Schenborn and Goiffin, Promega Notes 41:11, 1993). Luciferase activity assay kits are commercially available from, for example, Promega Corp., Madison, Wis. Target cell lines of this type can be used to screen libraries of chemicals, cell-conditioned culture media, fungal broths, soil samples, water samples, and the like. For example, a bank of cell-conditioned media samples can be assayed on a target cell to identify cells that produce ligand. Positive cells are then used to produce a cDNA library in a mammalian expression vector, which is divided into pools, transfected into host cells, and expressed. Media samples from the transfected cells are then assayed, with subsequent division of pools, re-transfection, subculturing, and re-assay of positive cells to isolate a cloned cDNA encoding the ligand.

A natural ligand for the ZCytor2 receptor can also be identified by mutagenizing a cell line expressing the receptor and culturing it under conditions that select for autocrine growth. See WIPO publication WO 95/21930. Within a typical procedure, BaF3 cells expressing ZCytor2 and human β_{c} are mutagenized, such as with 2-ethylmethanesulfonate (EMS). The cells are then allowed to recover in the presence of IL-3, then transferred to a culture medium lacking IL-3 and IL-4. Surviving cells are screened for the production of a ZCytor2 ligand, such as by adding soluble receptor to the culture medium or by assaying conditioned media on wild-type BaF3 cells and BaF3 cells expressing the receptor.

An additional screening approach provided by the present invention includes the use of hybrid receptor polypeptides. These hybrid polypeptides fall into two general classes. Within the first class, the intracellular domain of Z-Cytor2, comprising approximately residues 364 to 380 of SEQ ID NO:2, is joined to the ligand-binding domain of a second receptor. It is preferred that the second receptor be a hematopoietic cytokine receptor, such as mpl receptor (Souyri et al., Cell 63: 1137-1147, 1990). The hybrid receptor will further comprise a transmembrane domain, which may be derived from either receptor. A DNA construct encoding the hybrid receptor is then inserted into a host cell. Cells expressing the hybrid receptor are cultured in the presence of a ligand for the binding domain and assayed for a response. This system provides a means for analyzing signal transduction mediated by ZCytor2 while using readily available ligands. This system can also be used to determine if particular cell lines are capable of responding to signals transduced by ZCytor2. A second class of hybrid receptor polypeptides comprise the extracellular (ligand-binding) domain of ZCytor2 (approximately residues 25 to 337 of SEQ ID NO:2) with an intracellular domain of a second receptor, preferably a hematopoietic cytokine receptor, and a transmembrane domain. Hybrid receptors of this second class are expressed in cells known to be capable of responding to signals transduced by the second receptor. Together, these two classes of hybrid receptors enable the use of a broad spectrum of cell types within receptor-based assay systems.

Cells found to express the ligand are then used to prepare a cDNA library from which the ligand-encoding cDNA can be isolated as disclosed above. The present invention thus provides, in addition to novel receptor polypeptides, methods for cloning polypeptide ligands for the receptors.

The tissue specificity of ZCytor2 expression suggests a role in spermatogenesis, a process that is remarkably similar to the development of blood cells (hematopoiesis). Briefly, spermatogonia undergo a maturation process similar to the differentiation of hematopoietic stem cells. In both systems, the c-kit ligand is involved in the early stages of differentiation.

In view of the tissue specificity observed for this receptor, agonists (including the natural ligand) and antagonists have enormous potential in both in vitro and in vivo applications. Compounds identified as receptor agonists are useful for stimulating proliferation and development of target cells in vitro and in vivo. For example, agonist compounds are useful as components of defined cell culture media, and may be used alone or in combination with other cytokines and hormones to replace serum that is commonly used in cell culture. Agonists are thus useful in specifically promoting the growth and/or development of testis-derived cells in culture. Agonists and antagonists may also prove useful in the study of spermatogenesis and infertility. Antagonists are useful as research reagents for characterizing sites of ligand-receptor interaction. In vivo, receptor agonists may find application in the treatment of male infertility. Antagonists of receptor function may be useful as male contraceptive agents.

Zcytor2 receptor antagonists and ligand-binding polypeptides may also be used to modulate immune functions by blocking the action of IL-13. Of particular interest in this regard is the limiting of unwanted immune responses, such as allergies and asthma. Local administration is preferred to avoid systemic immune suppression. Examples of local administration include topical application to the skin and inhalation. Suitable methods of formulation are known in the art.

Zcytor2 may also be used within diagnostic systems for the detection of circulating levels of ligand. Within a related embodiment, antibodies or other agents that specifically bind to Zcytor2 can be used to detect circulating receptor polypeptides. Elevated or depressed levels of ligand or receptor polypeptides may be indicative of pathological conditions, including cancer.

ZCytor2 receptor polypeptides can be prepared by expressing a truncated DNA encoding residues 141 through 337 of a human Zcytor2 receptor (SEQ ID NO:2 or SEQ ID NO:4) or the corresponding region of a non-human receptor. Additional residues of the receptor may also be included, in particular amino-terminal residues between the predicted mature N-terminus (residue 25 of SEQ ID NO:2 or SEQ ID NO:4) and residue 141, and short C-terminal extensions. It is preferred that the extracellular domain polypeptides be prepared in a form substantially free of transmembrane and intracellular polypeptide segments. For example, the C-terminus of the receptor polypeptide may be at residue 338 or 339 of SEQ ID NO:2 or the corresponding region of an allelic variant or a non-human receptor. A preferred such polypeptide consists of residues 25 to 337 of SEQ ID NO:4. To direct the export of the receptor domain from the host cell, the receptor DNA is linked to a second DNA segment encoding a secretory peptide, such as a t-PA secretory peptide. To facilitate purification of the secreted receptor domain, a C-terminal extension, such as a poly-histidine tag, substance P, Flag™ peptide (Hopp et al., Biotechnology 6:1204-1210, 1988; available from Eastman Kodak Co., New Haven, Conn.) or another polypeptide or protein for which an antibody or other specific binding agent is available, can be fused to the receptor polypeptide.

In an alternative approach, a receptor extracellular domain can be expressed as a fusion with immunoglobulin heavy chain constant regions, typically an F_{c} fragment, which contains two constant region domains and a hinge region but lacks the variable region. Such fusions are typically secreted as multimeric molecules wherein the Fc portions are disulfide bonded to each other and two receptor polypeptides are arrayed in closed proximity to each other. Fusions of this type can be used to affinity purify the cognate ligand from solution, as an in vitro assay tool, to block signals in vitro by specifically titrating out ligand, and as antagonists in vivo by administering them parenterally to bind circulating ligand and clear it from the circulation. To purify ligand, a Zcytor2-Ig chimera is added to a sample containing the ligand (e.g., cell-conditioned culture media or tissue extracts) under conditions that facilitate receptor-ligand binding (typically near-physiological temperature, pH, and ionic strength). The chimera-ligand complex is then separated by the mixture using protein A, which is immobilized on a solid support (e.g., insoluble resin beads). The ligand is then eluted using conventional chemical techniques, such as with a salt or pH gradient. In the alternative, the chimera itself can be bound to a solid support, with binding and elution carried out as above. The chimeras may be used in vivo to induce infertility. Chimeras with high binding affinity are administered parenterally (e.g., by intramuscular, subcutaneous or intravenous injection). Circulating molecules bind ligand and are cleared from circulation by normal physiological processes. For use in assays, the chimeras are bound to a support via the Fc region and used in an ELISA format.

A preferred assay system employing a ligand-binding receptor fragment uses a commercially available biosensor instrument (BIAcore™, Pharmacia Biosensor, Piscataway, N.J.), wherein the receptor fragment is immobilized onto the surface of a receptor chip. Use of this instrument is disclosed by Karlsson, J. Immunol. Methods 145:229-240, 1991 and Cunningham and Wells, J. Mol. Biol. 234:554-563, 1993. A receptor fragment is covalently attached, using amine or sulfhydryl chemistry, to dextran fibers that are attached to gold film within the flow cell. A test sample is passed through the cell. If ligand is present in the sample, it will bind to the immobilized receptor polypeptide, causing a change in the refractive index of the medium, which is detected as a change in surface plasmon resonance of the gold film. This system allows the determination of on- and off-rates, from which binding affinity can be calculated, and assessment of stoichiometry of binding.

Ligand-binding receptor polypeptides can also be used within other assay systems known in the art. Such systems include Scatchard analysis for determination of binding affinity (see, Scatchard, Ann. NY Acad. Sci. 51: 660-672, 1949) and calorimetric assays (Cunningham et al., Science 253:545-548, 1991; Cunningham et al., Science 254:821-825, 1991).

A receptor ligand-binding polypeptide can also be used for purification of ligand. The receptor polypeptide is immobilized on a solid support, such as beads of agarose, cross-linked agarose, glass, cellulosic resins, silica-based resins, polystyrene, cross-linked polyacrylamide, or like materials that are stable under the conditions of use. Methods for linking polypeptides to solid supports are known in the art, and include amine chemistry, cyanogen bromide activation, N-hydroxysuccinimide activation, epoxide activation, sulfhydryl activation, and hydrazide activation. The resulting media will generally be configured in the form of a column, and fluids containing ligand are passed through the column one or more times to allow ligand to bind to the receptor polypeptide. The ligand is then eluted using changes in salt concentration or pH to disrupt ligand-receptor binding.

Zcytor2 polypeptides can also be used to prepare antibodies that specifically bind to Zcytor2 polypeptides. As used herein, the term “antibodies” includes polyclonal antibodies, monoclonal antibodies, antigen-binding fragments thereof such as F(ab′)₂ and Fab fragments, and the like, including genetically engineered antibodies. Antibodies are defined to be specifically binding if they bind to a Zcytor2 polypeptide with a Kₐ Of greater than or equal to 10⁷/M. The affinity of a monoclonal antibody can be readily determined by one of ordinary skill in the art (see, for example, Scatchard, ibid.).

Methods for preparing polyclonal and monoclonal antibodies are well known in the art (see for example, Sambrook et al., Molecular Cloning: A Laboratory Manual, Second Editiona, Cold Spring Harbor, N.Y., 1989; and Hurrell, J. G. R., Ed., Monoclonal Hybridoma Antibodies: Techniques and Applications, CRC Press, Inc., Boca Raton, Fla., 1982, which are incorporated herein by reference). As would be evident to one of ordinary skill in the art, polyclonal antibodies can be generated from a variety of warm-blooded animals such as horses, cows, goats, sheep, dogs, chickens, rabbits, mice, and rats. The immunogenicity of a Zcytor2 polypeptide may be increased through the use of an adjuvant such as Freund's complete or incomplete adjuvant. A variety of assays known to those skilled in the art can be utilized to detect antibodies which specifically bind to Zcytor2 polypeptides. Exemplary assays are described in detail in Antibodies: A Laboratory Manual, Harlow and Lane (Eds.), Cold Spring Harbor Laboratory Press, 1988. Representative examples of such assays include: concurrent immunoelectrophoresis, radio-immunoassays, radio-immunoprecipitations, enzyme-linked immunosorbent assays (ELISA), dot blot assays, inhibition or competition assays, and sandwich assays.

Antibodies to Zcytor2 are may be used for tagging cells that express the receptor, for affinity purification, within diagnostic assays for determining circulating levels of soluble receptor polypeptides, and as antagonists to block ligand binding and signal transduction in vitro and in vivo.

The invention is further illustrated by the following non-limiting examples.

### EXAMPLE 1

A cDNA library was prepared from human placental poly A⁺ RNA provided as a control in a Marathon™ cDNA Amplification Kit (Clontech, Palo Alto, Calif.) using the protocol provided by the manufacturer. This cDNA was used as template in polymerase chain reactions to generate DNA encoding human Zcytor2.

Primers were designed from the sequences of two expressed sequence tags (ESTs) in a DNA sequence database. Analysis of the EST sequences suggested that they represented the 5′ and 3′ ends of a cDNA encoding a cytokine receptor. One pair of primers, designated ZG9801 (SEQ ID NO:8) and ZG9941 (SEQ ID NO:9), were designed to be used in a 5′ RACE (rapid amplification of cDNA ends) reaction. A second pair, designated ZG9803 (SEQ ID NO:10) and ZG9937 (SEQ ID NO:11), were designed to be used in a 3′ RACE reaction. A third pair of primers, designated ZG9800 (SEQ ID NO:12) and ZG9802 (SEQ ID NO:13), were designed to amplify the region spanning the two ESTs. A fourth pair of primers, API (SEQ ID NO:14) and AP2 (SEQ ID NO:15), were supplied with the amplification kit or synthesized.

PCR amplification was carried out according to the instruction manual supplied with the kit, with certain modifications' to the protocol. For the 0.5° and 3′ RACE reactions, fifty pmol of each primer was used in each reaction. Each cDNA template was initially amplified using the appropriate gene-specific primer (ZG9801 or ZG9803) for 10 cycles. Primer AP1 was then added, and the reaction was continued for 25 cycles. The reaction mixture was incubated in a Hybaid OmniGene Temperature Cycling System (National Labnet Co., Woodbridge, N.Y.) for 1 minute at 95° C., then for 10 cycles of 60° C., 30 seconds; 72° C, 2 minutes; 95° C., 30 seconds. The mixture was held at 60° C., and 50 pmol of primer AP1 was added, and the reaction was continued for 25 cycles of 60° C., 30 seconds; 72° C., 2 minutes; 95° C., 30 seconds; followed by a 7 minute incubation at 72° C. The internal fragment was amplified under the same conditions using gene-specific primers (9800 and 9802), but AP1 was omitted. Reaction products were analyzed by electrophoresis on a 1% agarose gel. A discreet band was obtained for the internal fragment. The 5′ and 3′ RACE products were smears on the gel.

The 5′ and 3′ RACE products were purified using a PCR purification kit (Qiagen Inc., Chatsworth, Calif.) and used in nested PCR reactions. Each template was combined with 50 pmol of the appropriate specific primer (ZG9941 or ZG9937) and 50 pmol of primer. AP2. Reactions were run for 30 cycles of 95° C., 1 minute; 60° C., 30 seconds; 72° C., 3.5 minutes; then incubated at 72° C. for 7 minutes. The reaction products were analyzed by electrophoresis on a 1% agarose gel. One discreet band was obtained for each reaction.

The 5′ and 3′ products from the nested PCR reactions and the internal fragment from the initial Marathon™ PCR reaction were gel purified using a Qiagen Gel Extraction Kit.

The internal fragment was subcloned using a Stratagene (La Jolla, Calif.) pCR-Script™ SK(+) Cloning Kit according to the manufacturer's instructions, with 10 μl H₂O added to each reaction. The ligated DNA was then purified using CENTR1-SEP columns (Princeton Separations, Adelphia, N.J.) to increase the efficiency of transformation. The resulting vector was used to transform E. coli ElectroMAX DH10B™ cells (Gibco BRL, Gaithersburg, Md.) by electroporation.

Colonies were screened by PCR using gene-specific primers. Individual white colonies representing recombinants were picked and added to microcentrifuge tubes by swirling the toothpick with the colony on it in a tube containing 19.5 μl H₂O, 2.5 μl 10× Taq polymerase buffer (Boehringer Mannheim, Indianapolis, Ind.), 0.5 μl 10 mM dNTPs, 1.0 μl ZG9800 (SEQ ID NO:12) (20 pmol/μl), 10 μl ZG9802 (SEQ ID NO:13) (20 pmol/μl), and 0.5 μl Taq polymerase. Cells were streaked out on a master plate to use for starting cultures. Amplification reactions were incubated at 96° C. for 45 seconds to lyse the bacteria and expose the plasmid DNA, then run for 25 cycles of 96° C., 45 seconds; 55° C., 45 seconds; 72° C., 2 minutes to amplify cloned inserts. Products were analyzed by electrophoresis on a 1% agarose gel. One clone was identified as positive, and a plasmid template was prepared for sequencing using a QIAwell™ 8 Plasmid Kit (Qiagen Inc.).

The 5′ RACE product, the 3′ RACE product, the internal fragment and the internal fragment subclone were sequenced on an Applied Biosystems™ model 373 DNA sequencer (Perkin-Elmer Corporation, Norwalk, Conn.) using either an AmpliTaq® DyeDeoxy™ Terminator Cycle Sequencing Kit (Perkin-Elmer Corp.) or an ABI PRISM™ Dye Terminator Cycle Sequencing Core Kit (Perkin-Elmer Corp.). Oligonucleotides used in the PCR reactions were used as sequencing primers. In addition, primers ZG9850 (SEQ ID NO:16), ZG9851. (SEQ ID NO:17), ZG9852 (SEQ ID NO:18) and ZG9919 (SEQ ID NO:19) were used. Sequencing reactions were carried out in a Hybaid OmniGene Temperature Cycling System. Sequencher™ 3.0 sequence analysis software (Gene Codes Corporation, Ann Arbor, Mich.) was used for data analysis. Although the internal fragment subclone contained the entire coding sequence for the receptor, a composite sequence was constructed from all templates to include additional 5′ and 3′-untranslated sequence from the RACE products that was not present in the internal subclone. The full sequence is dislosed in SEQ ID NO:1.

A human cDNA was isolated by PCR using oligonucleotide primers specific for the gene sequence and containing restriction sites for subsequent manipulation of the DNA. Specific DNA was amplified from a human testis cDNA library using primers ZG10317 (SEQ ID NO:20) and ZG10319 (SEQ ID NO:21). 10 ng of template DNA was combined with 20 pmol of each primer, 5 μl of 10× buffer (Takara Shuzo Co., Ltd., Otsu, Shiga, Japan), 1 μl of ExTaq DNA polymerase (Takara Shuzo Co., Ltd.), and 200 μM dNTPs. The reaction was run for 30 cycles of 95° C., 30 seconds; 55° C., 30 seconds, and 68° C., 2 minutes; then incubated at 68° C. for 10 minutes. A fragment of approximately 1200 bp was recovered using a Wizard™ PCR Preps Purification System (Promega Corp., Madison, Wis.), cleaved with Xho I and Xba I, and a 1200 bp fragment was recovered by precipitation with ethanol.

The 1200 bp fragment was ligated into pHZ200, a vector comprising the mouse metallothionein-1 promoter, the bacteriophage T7 promoter flanked by multiple cloning banks containing unique restriction sites for insertion of coding sequences, the human growth hormone terminator, the bacteriophage T7 terminator, an E. coli origin of replication, a bacterial beta lactamase gene, and a mammalian selectable marker expression unit comprising the SV40 promoter and origin, a DHFR gene, and the SV40 transcription terminator. Plasmid pHZ200 was cleaved with Sal I and Xba I and was ligated to the Zcytor2 fragment.

The sequence of the human testis cDNA clone and the deduced amino acid sequence are shown in SEQ ID NO:3 and SEQ ID NO:4, respectively. The deduced amino acid sequence differs from that shown in SEQ ID NO:2 at residues 65, 180, and 259.

### EXAMPLE 2

Human Multiple Tissue Northern Blots (Human I, Human II, and Human III from Clontech) were probed to determine the tissue distribution of ZCytor2 expression. A probe was prepared by PCR. Single stranded DNA was prepared from K-562 mRNA (obtained from Clontech) using a RT-PCR kit (Stratagene Cloning Systems, La Jolla, Calif.) for use as template. 10 ng of template DNA was combined with 20 pmol of each of primers ZG9820 (SEQ ID NO:22) and ZG9806 (SEQ ID NO:23), 5 μl of 10× buffer (Clontech), 1 μl of KlenTaq DNA polymerase (Clontech), and 200 μM dNTPs. The reaction was run for 30 cycles of 95° C., 30 seconds; 55° C., 30 seconds, and 68° C., 2 minutes; then incubated at 68° C. for 10 minutes. The resulting DNA was purified by gel electrophoresis and ligated into pGEM®A/T (Promega Corp.). The resulting plasmid was used as a PCR template to generate the probe using the same reaction conditions described above for the K-562 template. DNA was purified by gel electrophoresis and labeled with ³²P by random priming. The blots were prehybridized in ExpressHyb™ hybridization solution (Clontech) at 65° C. for 1-6 hours, then hybridized in ExpressHyb™ solution containing 2×10⁶ cpm/ml of probe at 65° C. for from. 1.5 hour to overnight After hybridization the blots were washed at 50° C. in 0.1×SSC, 0.1% SDS. A transcript of approximately 1.5 kb was seen only in testis.

### EXAMPLE 3

A cDNA encoding a soluble human ZCytor2 receptor polypeptide was prepared by PCR. Human cDNA was prepared from a human testis cDNA library. DNA was amplified by PCR using 10 pmol each of oligonucleotide primers ZG10320 (SEQ ID NO:24.) and ZG10318 (SEQ ID NO:25). 10 ng of template DNA was combined with 20 pmol of each primers 5 μl of 10× buffer (Takara Shuzo Co., Ltd.), 1 μl of Taq DNA polymerase (Boehringer Mannheim), and 200 μM dNTPs. The reaction was run for 30 cycles of 95° C., 30 seconds; 55° C., 30 seconds, and 68° C., 2 minutes; then incubated at 68° C. for ten minutes. PCR products were separated by electrophoresis on a low melting point agarose gel (Boehringer Mannheim) and purified using a Wizard™ PCR Preps Purification System (Promega Corp.). The fragment was inserted into plasmid HSRT9 that had been cleaved with Bgl II and Xho I. HSRT9 is a mammalian cell expression vector derived from pHZ200 that contains a tissue plasminogen activator (t-PA) secretory signal sequence and a sequence encoding a C-terminal polyhistidine tag downstream of the MT-1 promoter. The resulting construct encoded a t-PA secretory peptide, human Zcytor2 residues 25—339 (SEQ ID NO:4), and a polyhistidine tag.

The soluble receptor expression vector is transfected into BHK 570 cells (ATCC No. CRL-10314) by liposome-mediated transfection (LIPOFECTAMINE™ Reagent, Life Technologies, Gaithersburg, Md.). Transfectants are cultured in the presence of methotrexate to select and amplify the transfected DNA. Soluble receptor polypeptide is recovered from conditioned culture media on nickel affinity purification columns (e.g., Talon spin columns from Clontech Laboratories). Columns are washed at neutral pH, and protein is eluted using a decreasing pH gradient or an imidazole gradient. Receptor monomers elute at about pH 6.0-6.3 of 50 mM imidazole, and receptor dimers elute at about pH 5.0-5.3 or 100 mM imidazole. In the alternative, batch purification can be employed.

### EXAMPLE 4

A cDNA library was prepared from a non-human primate. Testis tissue was obtained from a 13-year-old Celebus macaque. Total RNA was prepared from the tissue by the CsCl method (Chirgwin et al., Biochemistry 18:52-94, 1979). Poly(A)⁺ RNA was prepared from the total RNA by oligo(dT) cellulose chromatography (Aviv and Leder, Proc. Natl. Acad. Sci. USA 69:1408-1412, 1972). Double-stranded DNA was prepared from 1 μg of mRNA using a commercially available kit (Clontech Marathon™ cDNA amplification kit).

The macaque cDNA was amplified by PCR using a standard adapter-primer and primers derived from the human receptor cDNA sequence. Individual PCR mixtures (50 μl total volume) contained 5 μl template DNA, 5 μl 10× buffer (Clontech), 200 μM dNTPs (Perkin Elmer, CITY), 1 pl each of 10 pmol/μl primer APi (Clontech) and one of the primers (20 pmol/μl) shown in Table 4, and 1 μl of Klentaq-DNA polymerase (Clontech). The reactions were run for 3 cycles of 94° C., 30 seconds; 65° C., 30 seconds; 68° C., 30 seconds; 3 cycles of 94° C., 30 seconds; 60° C., 30 seconds; 68° C., 30 seconds; 3 cycles of 94° C., 30 seconds; 55′ C., 30 seconds; 68° C., 30 seconds; and 30 cycles of 94° C., 30 seconds; 500, 30 seconds; 68° C., 30 seconds; followed by a 68° C. incubation for 10 minutes.

TABLE 4

| | | Primer |
|---|---|---|
| Reaction No. | Primer No. | SEQ ID NO. |
| 1 | 9800 | 12 |
| 2 | 9820 | 22 |
| 3 | 9941 | 9 |
| 4 | 9801 | 8 |
| 5 | 9882 | 26 |
| 6 | 10082 | 27 |
| 7 | 9850 | 16 |
| 8 | 9919 | 16 |
| 9 | 10083 | 28 |
| 10 | 9803 | 10 |
| 11 | 10081 | 29 |
| 12 | 9881 | 30 |
| 13 | 9937 | 11 |
| 14 | 9806 | 23 |
| 15 | 9802 | 13 |

PCR products were electrophoresed on an agarose gel. The gel was stained with ethidium bromide and viewed under ultraviolet light. Bands from reactions amplified with primers 0.9800 and 9802 were of the expected size.

A second set of PCR reactions was run using the macaque cDNA (1:250 dilution) or first round PCR products from reactions 1, 2, 14 or 15 (Table 4) as templates the first round PCR products were purified using a Wizards PCR Preps Purification System (Promega Corp.) prior to use. 5 μl of template DNA was combined with other components as shown in Table 5. 1 μl of Klentaq DNA polymerase (Clontech) was added to each mixture. Reaction conditions were as specified above. Reaction products were electrophoresed on an agarose gel, stained with ethidium bromide, and visualized under UV light.

TABLE 5

| Rxn. | | 10x | | | | |
|---|---|---|---|---|---|---|
| No. | Template | Buffer | dNTPs | Primer 1 | Primer 2 | H₂O |
| 1 | macaque | 5 μl | 0.5 μl | — | — | 36.5 μl |
| 2 | macaque | 5 μl | 0.5 μl | 9800 | — | 36.5 μl |
| 3 | macaque | 5 μl | 0.5 μl | 9802 | — | 36.5 μl |
| 4 | macaque | 5 μl | 0.5 μl | 9800 | AP1 | 36.5 μl |
| 5 | macaque | 5 μl | 0.5 μl | 9802 | AP1 | 36.5 μl |
| 6 | macaque | 5 μl | 0.5 μl | AP1 | — | 36.5 μl |
| 7 | macaque | 5 μl | 0.5 μl | AP1 | 3′GP3DH | 36.5 μl |
| 8 | macaque | 5 μl | 0.5 μl | AP1 | 5′GP3DH | 36.5 μl |
| 9 | #14 | 5 μl | 0.5 μl | AP1 | 9806 | 36.5 μl |
| 10 | #15 | 5 μl | 0.5 μl | AP1 | 9802 | 36.5 μl |
| 11 | #1 | 5 μl | 0.5 μl | AP1 | 9800 | 36.5 μl |
| 12 | #2 | 5 μl | 0.5 μl | AP1 | 9820 | 36.5 μl |

Partial DNA and deduced amino acid sequences of macaque Zcytor2 cDNA are shown in SEQ ID NO:6 and SEQ ID NO:7. Alignment of the human and partial macaque sequences showed an amino acid sequence identity of 92% and a nucleotide sequence identity of 96% k.

### EXAMPLE 5

An expression vector encoding a human Zcytor2IgG fusion protein was constructed. The fusion comprised the extracellular domain of Zcytor2 fused at its C-terminus (residue 339 of SEQ ID NO:4) to the hinge region of the Fc portion of an IgG_{γ1} (Ellison et al., Nuc. Acids Res. 10:4071-4079, 1982). The hinge region was modified to replace a cysteine residue with serine to avoid unpaired cysteines upon dimerization of the fusion protein. A human t-PA secretory peptide was used to direct secretion of the fusion.

A human Zcytor2 DNA was prepared from a testis cDNA library by PCR using oligonucleotide primers ZG10320 (SEQ ID NO:24) and ZG10389 (SEQ ID NO:31). Twenty pmol of each primer was combined with 1 μl (10 ng) of template DNA, 10 μl of 2.5 mM dNTPs (Perkin-Elmer Corp.), 10 μl of 10× buffer (Klentaq PCR buffer, Clontech), 2 μl of Klentaq DNA polymerase (Clontech), and 70.8 μl H₂O. The reaction was run for 35 cycles of 94° C., 1 minute; 55° C., 1 minute; and 72° C., 2 minutes; followed by a 7 minute incubation at 72° C. The reaction products were extracted with phenol/CHCl₃, precipitated with ethanol, and digested with BglII. The DNA was electrophoresed on a agarose gel, and a 941 bp fragment was electrophoretically eluted from a gel slice, purified by phenol/CHCl₃ extraction, and precipitated with ethanol.

A human IgG_{γ1} clone was isolated from a human fetal liver cDNA library (Clontech) by PCR using oligonucleotide primers ZG10314 (SEQ ID NO:32) and ZG10315 (SEQ ID NO:33). The former primer introduced a BglII site into the hinge region (changing the third residue of the hinge region from Lys to Arg) and replaced the fifth residue of the hinge region. (Cys) with Ser. PCR was carried out essentially as described above for the Zcytor2 extracellular domain sequence. The DNA was digested with EcoRI and XbaI, and a 0.7 kb fragment was recovered by agarose gel electrophoresis, electroelution, phenol/CHCl₃ extraction, and ethanol precipitation. The IgG-encoding fragment and an XbaI-EcoRI linker were ligated into Zem229R (ATCC Accession No. 69447) that had been digested with EcoRI and treated with calf intestinal phosphatase. The resulting plasmid was digested with BglII and XbaI, and a 950 bp fragment was recovered by agarose gel electrophoresis, electroelution, phenol/CHCl₃ extraction, and ethanol precipitation.

To construct an expression vector for the Zcytor2-IgG fusion, a Zem229R vector containing a human t-PA secretory signal sequence joned to a human thrombopoietin sequence (disclosed in copending, commonly assigned U.S. patent application Ser. No. 08/347,029) was cleaved with BglII and XbaI. The fragment comprising the vector and t-PA secretory signal sequence was recovered and ligated to the IgG fragment. The Zcytor2 fragment was then ligated into this construct at the BglII site. The resulting plasmid was screened for the desired insert orientation. A plasmid with the desired orientation was designated h-Zcytor-2/IgG #709. Sequence analysis revealed a PCR-generated substitution resulting in an alanine codon instead of a valine codon at position 308 of SEQ ID NO:3.

Plasmid h-Zcytor-2/IgG was transfected into BHK-570 cells by liposome-mediated transfection (LIPOFECTAMINE™ Reagent, Life Technologies, Gaithersburg, Md.). Transfectants were cultured in medium containing 1 μM methotrexate for 10 days.

### EXAMPLE 6

The binding of ¹²⁵I-IL-13 to wild-type and Zcytor2-transfected BHK, TF-1, and BaF3 cells was determined. BHK cells were assayed in 6-well culture plates. TF-1 and BaF3 cells were assayed in microcentrifuge tubes. Cells were combined with 500 μl of either solution A (15 ml of binding buffer [RPMI containing 20 mM Tris pH7.4, 0.05% NaN₃, and 3 mg/ml BSA] plus 263 μl of ¹²⁵I-IL-13 [5.7×10⁷ cpm/ml]) or solution B (solution A containing 15 μl of cold 25 μg/ml IL-13). After a 2-hour incubation, cells were washed three times with 500 μl binding buffer and lysed in 500 μl of 400 mM NaOH. Lysates were transferred to tubes for gamma counting. BHK cells transfected to express Zcytor2 were found to specifically bind significant amounts of IL-13. In further experiments, binding of labeled IL-13 was found to be inhibited by IL-13 but not by IL-4.

Saturation binding analysis indicated that Zcytor2 expressed in BHK cells bound ¹²⁵I-IL-13 with a kd of 590±359 pM.

To determine if a soluble Zcytor2-IgG fusion could specifically bind IL-13, 1 μg of purified fusion protein was incubated in 200 μl of binding buffer containing 1 nM ¹²⁵I-IL-13±100 mM unlabeled IL-13 or IL-4. After two hours at room temperature with mixing, 25 μl of protein A-Sepharose was added, and the mixtures were incubated for an additional hour. The. Sepharose was washed three times and collected by centrifugation. Bound ¹²⁵I-IL-13 was determined by gamma counting. The fusion protein was found to bind significant amounts of labeled IL-13, which was blocked by excess unlabeled IL-13 but not by IL-4.

Binding of labeled IL-13 by BHK/Zcytor2 cells was measured in the presence and absence of the soluble Zcytor2-IgG fusion (0.005-5 ng/ml) or unlabeled IL-13. Binding was assayed essentially as described above. Both IL-13 and the fusion protein were found to inhibit binding of labeled IL-13 to the cells.

From the foregoing, it will be appreciated that, although specific embodiments of the invention have been described herein for purposes of illustration, various modifications may be made without deviating from the spirit and scope of the invention. Accordingly, the invention is not limited except as by the appended claims.

1. An isolated polynucleotide encoding a ligand-binding receptor polypeptide, said polypeptide comprising:
residues 141 to 337 of SEQ ID NO:2.

2. Cancelled.

3. An isolated polynucleotide according to claim 1 wherein said polypeptide further comprises a transmembrane domain.

4. An isolated polynucleotide according to claim 3 wherein said transmembrane domain comprises residues 340 to 363 of SEQ ID NO:2.

5. An isolated polynucleotide according to claim 3 wherein said polypeptide further comprises an intracellular domain.

6. An isolated polynucleotide according to claim 5 wherein said intracellular domain comprises residues 364 to 380 of SEQ ID NO:2.

7. An isolated-polynucleotide according to claim 1 wherein-said polypeptide comprises residues 25 to 337 of SEQ ID NO:2.

8. An isolated polynucleotide according to claim 1 wherein said polypeptide comprises residues 1 to 380 of SEQ ID NO:2.

9. An isolated polynucleotide according to claim 1 which is a DNA as shown in SEQ ID NO:1 from nucleotide 49 to nucleotide 1188.

10. An isolated polynucleotide according to claim 1 wherein said polypeptide further comprises an affinity tag.

11. An isolated polynucleotide according to claim 10 wherein said affinity tag is polyhistidine, protein A, glutathione S transferase, substance P, or an immunoglobulin heavy chain constant region.

12. An isolated polynucleotide according to claim 1 wherein said polynucleotide is DNA.

13. An expression vector comprising the following operably linked elements:
a transcription promoter;
a DNA segment encoding a secretory peptide and a ligand-binding receptor polypeptide, said polypeptide comprising
residues 141 to 337 of SEQ ID NO:2.

14. Cancelled.

15. An expression vector according to claim 13 wherein said polypeptide further comprises a transmembrane domain.

16. An expression vector according to claim 15 wherein said transmembrane domain comprises residues 340 to 363 of SEQ ID NO:2.

17. An expression vector according to claim 15 wherein said polypeptide further comprises an intracellular domain.

18. An expression vector according to claim 17 wherein said intracellular domain comprises residues 364 to 380 of SEQ ID NO:2.

19. An expression vector according to claim 13 wherein said polypeptide comprises residues 25 to 337 of SEQ ID NO:2.

20. An expression vector according to claim 13 wherein said polypeptide comprises residues 1 to 380 of SEQ ID NO:2.

21. An expression vector comprising the following operably linked elements:
(a) a transcription promoter;
(b) a DNA segment encoding a secretory peptide and a chimeric polypeptide, wherein said chimeric polypeptide consists essentially of a first portion and a second portion joined by a peptide bond, said first portion consisting essentially of a ligand binding domain of a receptor polypeptide and wherein said second portion is an affinity tag; and
(c) a transcription terminator.

22. An expression vector according to claim 21 wherein said affinity tag is an immunoglobulin Fc polypeptide.

23. A cultured eukaryotic cell into which has been introduced an expression vector according to claim 13, wherein said cell expresses a receptor polypeptide encoded by the DNA segment.

24. A cell according to claim 23 wherein said cell further expresses a hematopoietic receptor Pc subunit.

25. A cell according to claim 23 wherein said cell is dependent upon an exogenously supplied hematopoietic growth factor for proliferation.

26. An isolated polypeptide comprising
residues 141 to 337 of SEQ ID NO:2
wherein said polypeptide is substantially free of transmembrane and intracellular domains ordinarily associated with hematopoietic receptors.

27. A polypeptide according to claim 26 further comprising an immunoglobulin F_{c} polypeptide.

28. A polypeptide according to claim 26 further comprising an affinity tag.

29. A polypeptide according to claim 28 wherein said affinity tag is polyhistidine, protein A, glutathione S transferase, substance P, or an immunoglobulin heavy chain constant region.

30. A polypeptide according to claim 26 that is immobilized on a solid support.

31. A chimeric polypeptide consisting essentially of a first portion and a second portion joined by a peptide bond, wherein said first portion is a ligand binding domain of a receptor polypeptide
as shown in SEQ ID NO:2
and wherein said second portion is an affinity tag.

32. A polypeptide according to claim 31 wherein said affinity tag is an immunoglobulin F_{c} polypeptide.

33-39. Cancelled.
